(19) United States
(12) Patent Application Publication
(10) Pub. No.: US 2025/0325744 A1
(43) Pub. Date: Oct. 23, 2025

(54) Dosing System

(71) Applicant: LENUS INFUSIONSTECHNIK GMBH & CO.KG, Kiel (DE)

(72) Inventors: Rasmus KÖLLN, Schönkirchen (DE); Sven MARTSCHENKO, Kiel (DE); Uwe STUMPP, Frittlingen (DE)

(73) Assignee: LENUS INFUSIONSTECHNIK GMBH & CO.KG, Kiel (DE)

(21) Appl. No.: 18/875,463

(22) PCT Filed: Jun. 9, 2023
(86) PCT No.: PCT/DE2023/100436

§ 371 Date: Dec. 16, 2024

(30) Foreign Application Priority Data
Jul. 1, 2022 (DE) 10 2022 116 513.2

(51) Int. Cl. A61M 5/145; A61M 5/315

(52) U.S. Cl. CPC A61M 5/1456; A61M 5/31528

(57) ABSTRACT

The dosing system has a syringe with a syringe body having a nozzle, and a syringe plunger movably arranged in the syringe body and having a piston head; the dosing system also has a syringe pump that has a pump body, a syringe holder arranged on the pump body for mounting the syringe on the syringe pump, and a motor driving a threaded rod for driving the syringe plunger of the syringe, wherein the syringe plunger has a receptacle designed to receive at least one portion of the threaded rod and the threaded rod has a carriage which turns on the threaded rod and acts on the syringe plunger, characterized in that the carriage is designed to co-rotate with the threaded rod within a predetermined angular extent delimited respectively by a stop, and to be rotationally fixed when the carriage rests against the stop arranged in the rotational direction travelled by the threaded rod; and the syringe plunger and the carriage each have a connection means for forming a form fit connecting the syringe plunger and the carriage to each other, wherein the syringe plunger and the carriage are connected to each other when the carriage rests against the one stop, forming the form fit of the syringe plunger and the carriage, and are separated from each other when the carriage rests against the other stop.

The invention relates to a dosing system that can be used in particular as a mobile syringe pump. The invention relates specifically to a dosing system with a syringe which has a syringe body having a nozzle and a syringe plunger which is arranged displaceably in the syringe body and has a plunger head, and a syringe pump which has a pump body, a syringe holder arranged on the pump body for mounting the syringe on the syringe pump and a motor driving a threaded rod for driving the syringe plunger of the syringe, wherein the syringe plunger has a first receptacle arranged to receive at least a partial section of the threaded rod and the threaded rod has a slide screwing with the threaded rod and acting on the syringe plunger.

A dosing system of this type is known in particular from EP 1 219 312 A2 and DE 10 2020 121 665 A1.

The special feature of these dosing systems is that the plunger of the syringe is designed in such a way that the threaded rod is taken up by the plunger. Otherwise, the functional principle for pumping the fluid is identical to normal perfusers. The spindle is rotated around its own axis, whereby the slide, which is fixed on the spindle, drives the piston in the direction of the syringe nozzle and thus pushes the liquid out of the syringe. To ensure that the plunger moves in the syringe and the entire syringe is not pushed out of the pump body by the plunger, a bayonet lock is usually provided on the syringe pump to clamp the syringe body to the pump body. This ensures that the syringe itself is not moved, but rather the plunger inside the syringe.

One disadvantage of these dosing systems is that they are designed exclusively for emptying the syringes, but not for filling them. For example, separate filling aids are known for filling syringes that are to be used as part of the known dosing systems, with which the syringes are filled, whereby the filled syringe can then be inserted into the syringe pump for use in the dosing system.

The object of the invention is therefore to create a dosing system with which automated emptying and filling of the syringe with a syringe pump is possible.

According to the invention, this object is solved by the dosing system with the features of claim 1. The dependent claims disclose advantageous embodiments of the invention.

The basic idea of the invention is to oscillate the slide, which screws with the threaded rod and is usually mounted completely fixed in terms of rotation, at a predetermined angle so that it can rotate with the threaded rod over a predetermined distance. This limited rotary movement, which cannot cause the fluid picked up by the syringe to be conveyed, is used to form a detachable positive fit between the slide and the syringe plunger, whereby the stop provided for the slide in the respective direction of rotation ensures that the slide is mounted so that it cannot rotate when the threaded rod is driven further in the same direction of rotation. However, this enables the slide to automatically pull the syringe plunger into the pump body and the syringe to be filled with a fluid, in particular a medication, in just one device.

When the direction of rotation is reversed to empty the syringe, the positive locking is cancelled so that the slide—which in turn is mounted on a stop provided in the opposite direction to prevent rotation—pushes the syringe plunger out of the pump body into the syringe and the syringe as a whole can be easily removed when empty.

The advantage of the present invention therefore lies in the fact that a syringe used with a syringe pump can be inserted into the syringe pump when empty and filled and emptied with the syringe pump without the need for a further device for filling the syringe. The dosing system according to the invention is thus particularly suitable for easy handling by the supply of medication by means of the dosing systems known per se.

According to the invention, a dosing system is thus proposed with a syringe which has a syringe body having a nozzle and a syringe plunger which is arranged displaceably in the syringe body and has a plunger head, and a syringe pump which has a pump body a syringe holder arranged on the pump body for mounting the syringe on the syringe pump and a motor driving a threaded rod for driving the syringe plunger of the syringe, wherein the syringe plunger has a first receptacle arranged to receive at least a partial section of the threaded rod and the threaded rod has a slide screwing with the threaded rod and acting on the syringe plunger. The plunger head is designed in particular as a flange at the end of the syringe plunger in the form of a thumb rest. The dosing system according to the invention is further designed in such a way that the slide rotates with the threaded rod at a predetermined angle limited by a stop in each case and is fixed against rotation when the slide comes into contact with the stop arranged in the direction of rotation exerted by the threaded rod. In addition, the syringe plunger and the slide each have a connecting means for forming a positive fit connecting the syringe plunger and slide to one another, so that the syringe plunger and the slide are connected to one another forming the positive fit of the syringe plunger and slide when the slide is in contact with one stop and are separated from one another when the slide is in contact with the other stop.

Due to this design, it is possible for the slide to perform a limited rotary movement which is used to form and separate a positive fit between the slide and syringe plunger, whereby the stops formed by the syringe pump and limiting the rotary movement ensure that the slide is mounted so that it cannot rotate.

Preferably, the syringe plunger has a recess designed to accommodate the connecting means formed on the slide. This can, for example, be an opening provided on the syringe plunger, in particular on the plunger head, into which the connecting means provided on the slide can enter and be secured by means of rotation. The connecting means formed on the slide is particularly preferably a latching means. In particular, the connecting means formed on the slide is a hook. In any case, the connecting means are designed in such a way that the mutual displacement of the slide and syringe plunger in the axial direction of the syringe plunger is prevented when a positive fit exists. Rather, a movement of the slide is transferred directly to the syringe plunger when a positive fit exists, so that the syringe can be filled in particular. However, a positive fit is not required for emptying the syringe, as the syringe plunger only needs to be pushed off the slide for emptying.

According to a highly preferred embodiment, the connecting means arranged on the slide has, on its side facing the syringe plunger, a guide structure which co-operates with the connecting means arranged on the syringe plunger and is set up to position the slide in a position preparatory to the formation of the positive fit. This can be an incline which, for example, results in a connecting means designed as a hook on the slide being positioned in such a way that the hook can enter an opening provided on the syringe plunger.

Finally, the angular dimension at which the slide can perform a pendulum movement is preferably ≤25° and particularly preferably ≤20°. In any case, the pendulum movement is dimensioned in such a way that a positive locking caused by the pendulum movement can also be completely cancelled when the direction of rotation of the threaded rod is reversed and the syringe can be safely removed from the pump.

The invention is explained in more detail below with reference to a particularly preferably designed embodiment shown in the accompanying drawings. These show:

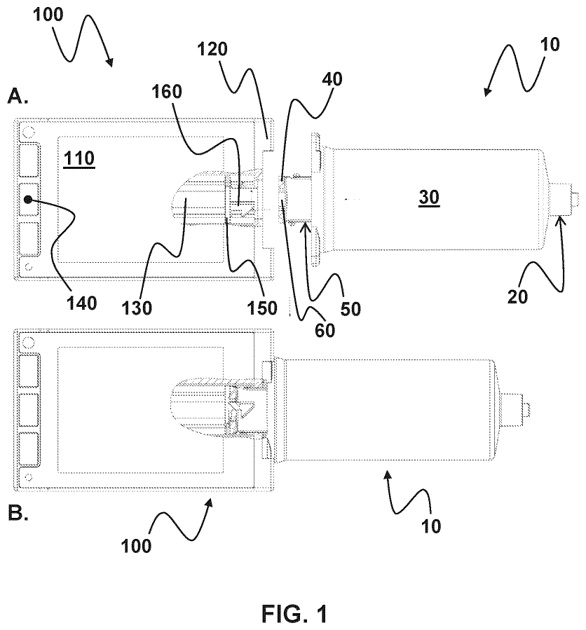

FIG. 1 a partially sectioned view of the dosing system consisting of syringe and syringe pump in a separated state (A) and in a connected state (B);

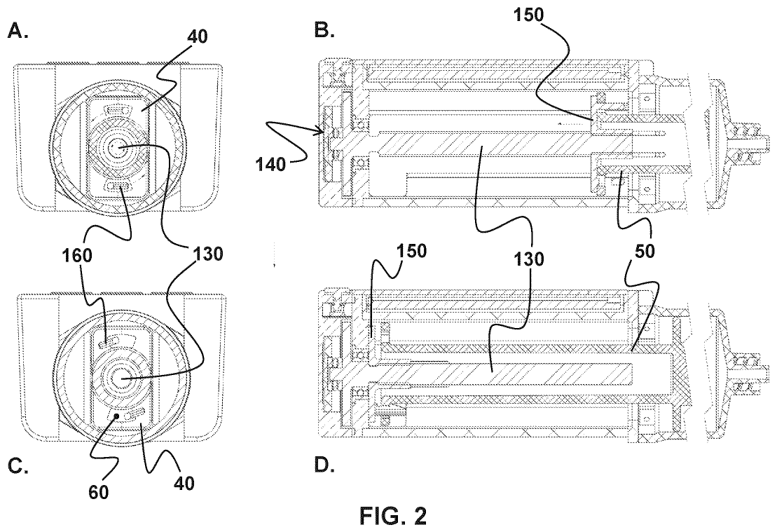

FIG. 2 the process of filling the syringe by means of the preferably designed dosing system at the beginning of the filling process in a cross-section at the level of the connection between the slide and the syringe plunger (A) and in a longitudinal section (B) and after completion of the filling process in a cross-section at the level of the connection between the slide and the syringe plunger (C) and in a longitudinal section (D);

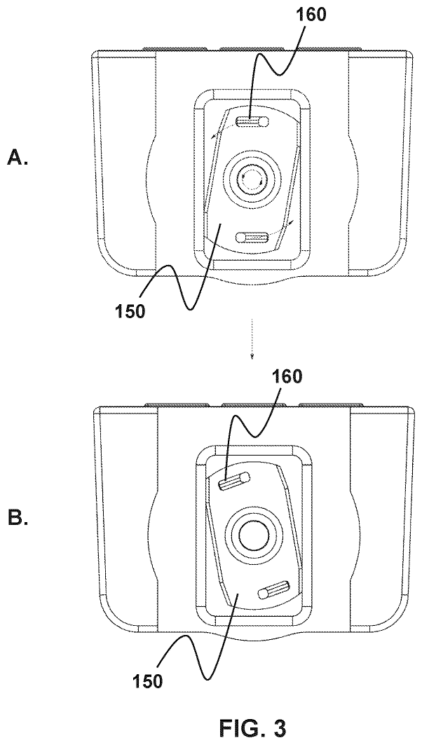

FIG. 3 the oscillating movement of the slide from a position in which the slide is not engaged with the syringe plunger (A) to a position locking the syringe plunger in the syringe pump (B);

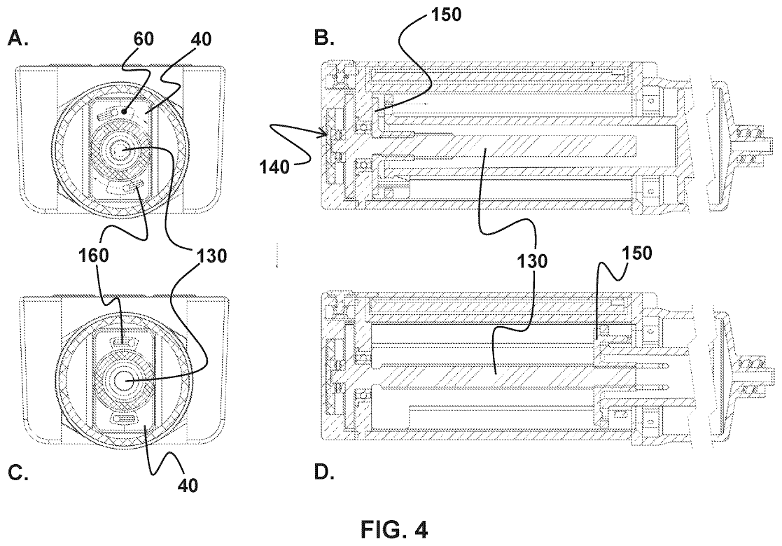

FIG. 4 the process of emptying the syringe by means of the preferably designed dosing system before emptying in a cross-section at the level of the connection between the slide and the syringe plunger (A) and in a longitudinal section (B) and towards the end of emptying in a cross-section at the level of the connection between the slide and the syringe plunger (C) and in a longitudinal section (D); and

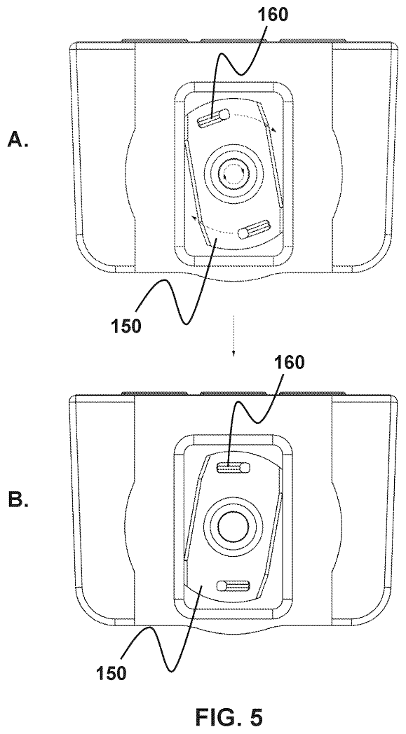

FIG. 5 the oscillating movement of the slide from the position (A) locking the syringe plunger in the syringe pump to the position (B) releasing the syringe plunger.

FIG. 1 shows a partially sectioned view of the dosing system consisting of syringe and syringe pump in a separate state (A) and in a connected state (B). In particular, FIG. 1 shows a particularly preferably designed dosing system consisting of a syringe 10 and a syringe pump 100.

The syringe 10 has a syringe body 30 formed with a nozzle 20, in which a syringe plunger 50 having a plunger head 40 is displaceably arranged.

The syringe pump 100 has a pump body 110 with a syringe holder 120 arranged on the pump body 110, which is set up for mounting the syringe 10 on the syringe pump 100. Furthermore, the syringe pump 100 has a threaded rod 130 driven by a motor 140, which is set up to drive the syringe plunger 50 of the syringe 10. For this purpose, the syringe plunger 50 is equipped with a receptacle designed to receive at least a partial section of the threaded rod 130 and the threaded rod 130 is equipped with a slide 150 which screws onto the threaded rod 130 and acts on the syringe plunger 50.

In contrast to the known systems, in which the slide is set up for exclusive movement in an axial direction—with reference to the longitudinal axis of the threaded rod 130—the slide 150 is set up to rotate with the threaded rod 130 at a predetermined angle, limited in each case by a stop, and is only fixed against rotation when the slide 150 is in contact with the stop arranged in the direction of rotation exerted by the threaded rod 130.

With the aid of the connecting means 60, 160 provided on the syringe plunger 50 and on the slide 150 for forming a positive fit connecting the syringe plunger 50 and the slide 150 to one another, a first position is thus assumed in which the syringe plunger 50 and the slide 150 are connected to one another, forming the positive fit of the syringe plunger 50 and the slide 150, when the slide 150 is in contact with the one stop, and a second position is made possible in which the syringe plunger 50 and the slide 150 are separated from one another when the slide 150 is in contact with the other stop.

As the following figures show, this design enables both the automated filling of the syringe 10 by pulling the syringe plunger 50 into the pump body 110 by means of the slide 150 and the automated emptying of the syringe 10 by means of the slide 150 as well as the unhindered removal of the syringe 10 from the pump body 110.

Firstly, FIG. 2 shows the process of filling the syringe by means of the preferably designed dosing system at the beginning of the filling process in a cross-section at the level of the connection between slide 150 and syringe plunger 50 (FIG. 2A) and in a longitudinal section (FIG. 2B) as well as after completion of the filling process in a cross-section at the level of the connection between slide 150 and syringe plunger 50 (FIG. 2C) and in a longitudinal section (FIG. 2D).

The syringe 10 is inserted into the syringe pump 100 and fixed to the pump body 110 in a rotationally and positionally fixed manner by means of a syringe holder 120 specially designed as a bayonet lock. The piston head 40 of the syringe plunger has a recess 60 as a connecting means 60 for receiving the connecting means 160 formed on the slide 150. The connecting means 160 arranged on the slide 150 has, on its side facing the syringe plunger 50, a guide structure which co-operates with the connecting means 60 arranged on the syringe plunger 50 and is set up to position the slide 150 in a position preparatory to the formation of the positive fit. This guiding structure is designed in particular as an incline, so that—if the slide 150 is not in a suitable starting position for forming the positive fit—the slide 150 can be rotated into the suitable (starting) position by the action of the syringe plunger 50 on the connecting means 160, in which the connecting means 160 can enter the recess 60 formed on the plunger head 40.

If the slide—as indicated in FIG. 2A—is now rotated anti-clockwise in the present case, a connection between the slide 150 and the syringe plunger 50 is created by means of positive locking between the connecting means 60, 160, which allows the syringe plunger 50 to be pulled into the syringe barrel 110.

The axial movement of the slide 150 along the threaded rod 130 is ensured—as FIG. 3 shows—by the fact that the slide 150, which has the connecting means 160, can rotate with the threaded rod 130 at a predetermined angle when the latter is driven until the slide 150 abuts against a stop formed by the pump body 110 and, when the threaded rod 130 continues to rotate in the same direction of rotation, is mounted in a non-rotatable manner and moves along the longitudinal axis of the threaded rod 130. During this axial movement of the slide 150, the slide takes the syringe plunger 50 with it due to the positive fit created by the slide 150 and the syringe plunger 50, so that the syringe 10 can be drawn up and the syringe 10 can be filled.

The syringe emptying process—which is shown in FIG. 4 and FIG. 5—now takes place in reverse order. The threaded rod 130 is driven in the opposite direction to the previous figures, so that the slide 150 now rotates with the threaded rod 130 in the opposite direction at the specified angular dimension until the stop provided in this direction of rotation is reached and the slide 150 is mounted so that it cannot rotate. In the example shown, the entire angular range covered by the oscillating movement of the slide 150 is approximately 20°.

1. A dosing system comprising:
a syringe which has a syringe body having a nozzle and a syringe plunger which is arranged displaceably in the syringe body and has a plunger head, and
a syringe pump which has a pump body, a syringe holder arranged on the pump body for mounting the syringe on the syringe pump and a motor driving a threaded rod for driving the syringe plunger of the syringe,
wherein the syringe plunger has a receptacle arranged to receive at least a partial section of the threaded rod and the threaded rod has a slide screwing with the threaded rod and acting on the syringe plunger,
the slide rotates with the threaded rod at a predetermined angular dimension limited by a respective stop and is fixed against rotation when the slide abuts against the stop arranged in the direction of rotation exerted by the threaded rod,
the syringe plunger and the slide each have a connector connecting means for forming a positive fit connecting the syringe plunger and slide to one another, and
the syringe plunger and the slide are connected to one another, forming the positive fit of syringe plunger and slide, when the slide is in contact with the one stop and are separated from one another when the slide is in contact with the other stop.

2. The dosing system according to claim 1, wherein the syringe plunger has a recess formed on the slide designed to receive the connector.

3. The dosing system according to claim 1, wherein the connector formed on the slide is a latch.

4. The dosing system according to claim 1, wherein the connector formed on the slide is a hook.

5. The dosing system according to claim 1, wherein the connector arranged on the slide has, on its side facing the syringe plunger, a guide structure cooperating with the connector arranged on the syringe plunger and set up for positioning the slide in a position preparing for formation of the positive fit.

6. The dosing system according to claim 1, wherein the angular dimension is ≤25°.

7. The dosing system according to claim 2, wherein the connector formed on the slide is a latch.

8. The dosing system according to claim 2, wherein the connector formed on the slide is a hook.

9. The dosing system according to claim 2, wherein the connector arranged on the slide has, on its side facing the syringe plunger, a guide structure cooperating with the connector arranged on the syringe plunger and set up for positioning the slide in a position preparing for formation of the positive fit.

10. The dosing system according to claim 2, wherein the angular dimension is ≤25°.

11. The dosing system according to claim 3, wherein the connector arranged on the slide has, on its side facing the syringe plunger, a guide structure cooperating with the connector arranged on the syringe plunger and set up for positioning the slide in a position preparing for formation of the positive fit.

12. The dosing system according to claim 3, wherein the angular dimension is ≤25°.

13. The dosing system according to claim 4, wherein the connector arranged on the slide has, on its side facing the syringe plunger, a guide structure cooperating with the connector arranged on the syringe plunger and set up for positioning the slide in a position preparing for formation of the positive fit.

14. The dosing system according to claim 4, wherein the angular dimension is ≤25°.
